(19) United States
(12) Patent Application Publication
(10) Pub. No.: US 2026/0261539 A1
(43) Pub. Date: Sep. 3, 2026

(54) SYSTEM

(71) Applicant: SOFTBANK GROUP CORP., Tokyo (JP)

(72) Inventor: Tamon SASAKI, Tokyo (JP)

(73) Assignee: SOFTBANK GROUP CORP., Tokyo (JP)

(21) Appl. No.: 19/554,711

(22) Filed: Mar. 3, 2026

Related U.S. Application Data
(60) Provisional application No. 63/766,188, filed on Mar. 3, 2025.

(51) Int. Cl. H04L 51/216; G06F 40/30; G16H 40/67

(52) U.S. Cl. CPC H04L 51/216; G06F 40/30; G16H 40/67

(57) ABSTRACT

A system for efficiently reviewing interactions between users within a messenger application is disclosed. This system comprises a data collection unit, a data analysis unit, a summary generation unit, a randomization unit, and a customization unit. The data collection unit collects message data with user permission and transmits it to a server. The data analysis unit analyzes messages using natural language processing technology to identify conversation themes and sentiments. The summary generation unit uses a generative AI model to summarize conversations in narrative form. The randomization unit randomly selects summaries and provides them to users. The customization unit allows users to generate summaries by specifying particular conditions. This enables users to review past conversations from new perspectives and deepen emotional connections.

### CROSS-REFERENCE TO RELATED APPLICATION

This application claims priority under 35 U.S.C. § 119 to U.S. Provisional Patent Application No. 63/766,188, filed on March 3, 2025, the entire contents of which are incorporated herein by reference.

### BACKGROUND

### Technical Field

The present disclosure relates to a system.

### Related Art

Japanese Patent Application Publication Laid-Open (JP-A) No. 2022-180282 discloses a persona chatbot control method performed by at least one processor, comprising: a step of receiving a user utterance; a step of adding to the user utterance a prompt containing a description of the chatbot's persona and related instructions; a step of encoding the prompt; and a step of inputting the encoded prompt into a language model to generate a chatbot utterance in response to the user utterance.

### SUMMARY

Modern communication often occurs via text messages, and the sheer volume of daily exchanges makes manually scrolling back to review past conversations extremely cumbersome and time-consuming for users. Furthermore, when reviewing past conversations, users risk missing important points or emotionally significant moments. Additionally, when users wish to focus on specific conversations or time periods to review past exchanges, current messenger apps do not sufficiently provide such functionality. It is desired that users may efficiently review past conversations from a new perspective and deepen emotional connections by automatically summarizing chat histories using generative AI and presenting them in a narrative format. It is desired that users are provided with new discoveries and surprises by offering summaries randomly, thereby sparking renewed interest in past conversations. Furthermore, the paid version seeks to enable flexible review tailored to user needs and provide a more personalized experience by offering a feature to generate summaries for specific contacts or time periods.

As a means to solve the problem, a system is provided comprising: a data collection unit that collects message data; a data analysis unit that analyzes the collected message data and extracts important information; a summary generation unit that generates a story-format summary based on the extracted information; a randomization unit that randomly selects and provides the generated summary to the user; and a customization unit that provides a customization function allowing the user to generate summaries by specifying specific conditions. The data collection unit obtains data including text messages, sender IDs, recipient IDs, and timestamps via the messenger app's API with user permission and securely stores the acquired data in encrypted format. The data analysis unit analyzes the message data using natural language processing technology, performs morphological analysis to understand the conversation structure, and uses keyword extraction algorithms to identify conversation themes and important topics.

The summary generation unit uses generative AI models to understand the flow of the conversation, extract key points, and generate narrative-style text. The randomization unit randomly selects the summarized narratives, to provide users with new discoveries and surprises. The Customization Unit, available in the paid version, enables users to generate summaries for specific individuals or time periods, allowing flexible reflection tailored to user needs. This enables users to efficiently revisit past conversations and deepen emotional connections.

### BRIEF DESCRIPTION OF THE DRAWINGS

FIG. 1 is a conceptual diagram showing an example configuration of the data processing system according to the first embodiment.

FIG. 2 is a conceptual diagram showing an example of key functions of the data processing device and smart device according to the first embodiment.

FIG. 3 is a conceptual diagram showing an example configuration of the data processing system according to the second embodiment.

FIG. 4 is a conceptual diagram showing an example of the main functions of the data processing device and smart glasses according to the second embodiment.

FIG. 5 is a conceptual diagram showing an example configuration of a data processing system according to a third embodiment.

FIG. 6 is a conceptual diagram showing an example of the main functions of the data processing device and headset terminal according to the third embodiment.

FIG. 7 is a conceptual diagram showing an example configuration of a data processing system according to the fourth embodiment.

FIG. 8 is a conceptual diagram showing an example of the main functions of the data processing device and robot according to the fourth embodiment.

FIG. 9 shows an emotion map onto which multiple emotions are mapped.

FIG. 10 shows an emotion map onto which multiple emotions are mapped.

### DETAILED DESCRIPTION

Hereinafter, embodiments of a data processing device, a data processing method, and a program according to the technology of the disclosure will be described with reference to the accompanying drawings.

The following describes an example embodiment of a system according to the present disclosure with reference to the accompanying drawings.

First, the terminology used in the following description is explained.

In the following embodiments, a signed processor (hereinafter simply referred to as a "processor") may be a single arithmetic unit or a combination of multiple arithmetic units. Furthermore, the processor may be a single type of arithmetic unit or a combination of multiple types of arithmetic units. Examples of processing units include a CPU (Central Processing Unit), GPU (Graphics Processing Unit), GPGPU (General-Purpose computing on Graphics Processing Units), and APU (Accelerated Processing Unit).

In the following embodiments, signed RAM (Random Access Memory) is a memory where information is temporarily stored and is used as working memory by the processor.

In the following embodiments, the signed storage is one or more non-volatile storage devices that store various programs and various parameters, etc. Examples of non-volatile storage devices include flash memory (SSD (Solid State Drive)), magnetic disks (e.g., hard disk), or magnetic tape.

In the following embodiments, the communication I/F (Interface) with code refers to an interface including a communication processor and an antenna, among others. The communication I/F governs communication between multiple computers. Examples of communication standards applicable to the communication I/F include wireless communication standards such as 5G (5th Generation Mobile Communication System), Wi-Fi (registered trademark), or Bluetooth (registered trademark).

In the following embodiments, "A and/or B" is synonymous with "at least one of A and B." That is, "A and/or B" means it may be only A, only B, or a combination of A and B. Furthermore, in this specification, when three or more items are expressed connected by "and/or," the same concept applies as for "A and/or B".

### First Embodiment

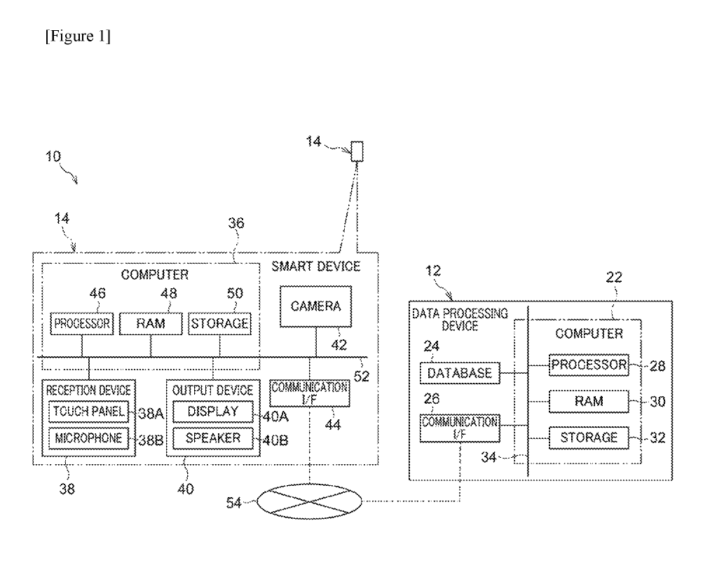

FIG. 1 shows an example configuration of a data processing system 10 according to the first embodiment.

As shown in FIG. 1, the data processing system 10 includes a data processing device 12 and a smart device 14. An example of the data processing device 12 is a server.

The data processing device 12 includes a computer 22, a database 24, and a communication I/F 26. The computer 22 is an example of a "computer" according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The smart device 14 includes a computer 36, a reception device 38, an output device 40, a camera 42, and a communication I/F 44. The computer 36 includes a processor 46, RAM 48, and storage 50. The processor 46, RAM 48, and storage 50 are connected to a bus 52. The reception device 38, output device 40, and camera 42 are connected to the bus 52. The reception device 38, output device 40, and camera 42 are also connected to the bus 52.

The reception device 38 includes a touch panel 38A and a microphone 38B, among other components, and receives user input. The touch panel 38A receives user input via contact with an indicator (e.g., a pen or finger) by detecting such contact. The microphone 38B receives voice-based user input by detecting the user's voice. The control unit 46A transmits data indicating the user input received via the touch panel 38A and microphone 38B to the data processing unit 12. Within the data processing unit 12, the specific processing unit 290 acquires the data indicating the user input.

Output device 40 includes display 40A and speaker 40B, among others, presenting data to user 20 by outputting it in a perceptible form (e.g., audio and/or text). Display 40A displays visual information such as text and images according to instructions from processor 46. Speaker 40B outputs audio according to instructions from processor 46 Camera 42 is a compact digital camera equipped with an optical system including a lens, aperture, and shutter, and an imaging element such as a CMOS (Complementary Metal-Oxide-Semiconductor) image sensor or a CCD (Charge Coupled Device) image sensor.

The communication interface 44 is connected to the network 54. The communication interfaces 44 and 26 manage the exchange of various information between processor 46 and processor 28 via network 54.

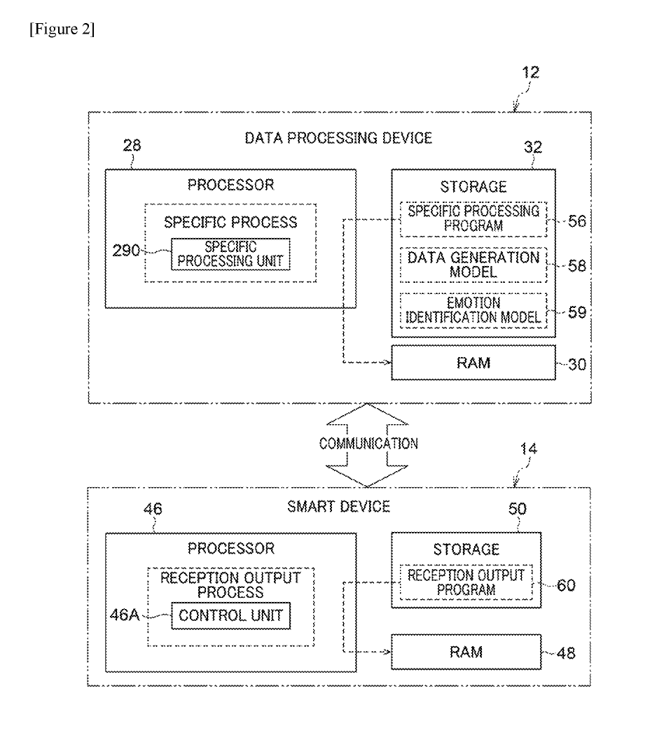

FIG. 2 shows an example of the main functions of the data processing device 12 and the smart device 14.

As shown in FIG. 2, specific processing is performed by processor 28 in data processing device 12. Specific processing program 56 is stored in storage 32. Specific processing program 56 is an example of a "program" related to the technology of this disclosure. The processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and emotion identification model 59 are used by specific processing unit 290. Specific processing unit 290 can estimate a user's emotion using emotion identification model 59 and perform specific processing using the user's emotion. The emotion estimation function (emotion identification function) using the emotion identification model 59 performs various estimations and predictions concerning the user's emotion, including estimation and prediction of the user's emotion, but is not limited to such examples. Furthermore, estimation and prediction of emotion also includes, for example, analysis (parsing) of emotion.

The smart device 14 performs reception output processing via the processor 46. The reception output program 60 is stored in the storage 50. The reception output program 60 is used in conjunction with the specific processing program 56 by the data processing system 10. The processor 46 reads the reception output program 60 from the storage 50 and executes the read reception output program 60 on the RAM 48. The specific processing is performed by the processor 46 operating as a control unit 46A according to the specific processing program 60 executed on the RAM 48. Note that the smart device 14 may also have data generation models and emotion identification models similar to the data generation model 58 and emotion identification model 59, and may perform processing similar to that of the specific processing unit 290 using these models. The reception output processing is realized by the processor 46 operating as the control unit 46A according to the reception output program 60 executed on the RAM 48.

Other devices besides the data processing device 12 may also have the data generation model 58. For example, a server device (e.g., a generation server) may have the data generation model 58. In this case, the data processing device 12 communicates with the server device having the data generation model 58 to obtain processing results (such as prediction results) obtained using the data generation model 58. Furthermore, the data processing device 12 may be the server device itself, or it may be a terminal device owned by a user (e.g., a mobile phone, robot, home appliance, etc.) owned by a user. Next, an example of processing by the data processing system 10 according to the first embodiment will be described.

### Example 1

The flow of the specific processing in Example 1 is described below. The components of the system described below are implemented by the data processing device 12 and the smart device 14. The data processing device 12 is referred to as the "server," and the smart device 14 is referred to as the "terminal."

### Implementation Examples for Carrying Out the Invention

The system configuration using the server and terminal for implementing the present invention is described in further detail below.

First, the data collection unit is described. This unit operates via the API of a messenger application installed on the user's terminal. When the user launches the app, the data collection unit confirms the user's permission and only begins collecting message data if permission is granted. Specifically, it retrieves the history of the chat thread selected by the user and collects information containing metadata such as text messages, sender ID, recipient ID, and timestamps. This data is temporarily stored within the terminal and encrypted using encryption technology such as AES. The encrypted data is then transmitted to the server using a secure communication protocol (e.g., TLS) to the server.

Next, the data analysis unit operates on the server. This unit decrypts the received encrypted data and performs analysis using natural language processing (NLP) technology. Specifically, it uses a morphological analysis engine to parse the grammatical structure of each message and identify parts of speech such as nouns, verbs, and adjectives. Furthermore, it applies a keyword extraction algorithm to extract frequently occurring words and phrases within the conversation. For example, it identifies words related to specific events or emotions to grasp the conversation's theme. Additionally, it uses sentiment analysis technology to evaluate the emotional tone of each message, assigning sentiment labels such as positive, negative, or neutral.

The summary generation unit also operates on the server, generating summaries based on information obtained from the data analysis unit. This unit uses generative AI models, such as transformer models, to understand the flow of the conversation and extract key points. The AI model emphasizes particularly emotional moments or points where important decisions were made within the conversation and generates narrative-style text. For example, if users are discussing a specific event, it constructs a story centered around messages related to that event. Furthermore, the AI considers the flow of the conversation from start to finish to form a natural storyline.

The randomization unit has the function of randomly selecting generated summaries and runs on the server. This unit uses a random number generation algorithm to randomly select the summary provided to the user. For example, it ensures a different story is displayed each time the user opens the app, providing new discoveries and surprises. During randomization, it considers the user's past viewing history to prevent the same story from being displayed too frequently.

The Customization Unit is implemented as a user interface on the terminal. This unit provides functionality in the paid version for users to generate summaries by specifying particular individuals or time periods. For example, if a user wishes to review conversations with a specific friend or interactions from a particular month, they can set conditions using a dropdown menu or calendar widget. Once the user sets the conditions, this information is sent to the server, and a summary is generated based on the specified conditions.

Finally, the generated story is sent to the device and displayed through the user interface. Users can browse the story and review past interactions via an interface designed to be intuitive and easy to use. For example, it provides a viewer allowing users to scroll and read the story, and a function to save specific stories as favorites. Users can also share the story on social media or other messenger apps.

Thus, the present invention is realized through cooperation between the server and the terminal, providing users with an efficient and emotionally resonant reflection experience. The entire system operates seamlessly, enabling flexible services tailored to user needs.

### System Configuration

The system according to this embodiment comprises a data collection unit, a data analysis unit, a summary generation unit, a randomization unit, and a customization unit. The data collection unit has the function of collecting message data via the API of a messenger application installed on the user's terminal, after obtaining the user's permission. When a user selects a specific chat thread, this unit retrieves its history and collects information including metadata such as text messages, sender ID, recipient ID, and timestamps. For example, if a user wishes to review past conversations with a friend, the history of that conversation is collected. Data is temporarily stored within the terminal, encrypted using encryption techniques such as AES, and transmitted to the server via a secure communication protocol.

The data analysis unit runs on the server, decrypts the received encrypted data, and performs analysis using natural language processing technology. This unit uses a morphological analysis engine to parse the grammatical structure of each message and identify parts of speech such as nouns, verbs, and adjectives. For example, it extracts frequently occurring words and phrases within a conversation to grasp the conversation's theme. It also uses sentiment analysis technology to evaluate the emotional tone of each message, assigning sentiment labels such as positive, negative, or neutral. Furthermore, it applies keyword extraction algorithms to identify words related to specific events or emotions, thereby pinpointing the central topic of the conversation.

The summary generation unit operates on the server and generates summaries based on information obtained by the data analysis unit. This unit uses a generative AI model to understand the flow of the conversation and extract key points. The AI model emphasizes particularly emotional moments or points where important decisions were made within the conversation and generates narrative-style text. For example, if users are discussing a specific event, it constructs a narrative centered around messages related to that event. Furthermore, the AI considers the flow from the start to the end of the conversation to form a natural storyline. Specific examples of prompt sentences fed to the generative AI include: "Highlight the most emotional moments in this conversation and summarize them as a story," or "Extract the key points of this conversation and express them in narrative form."

The randomization unit has the function of randomly selecting generated summaries and runs on the server. This unit uses a random number generation algorithm to randomly select the summary provided to the user. For example, it ensures a different story is displayed each time the user opens the app, providing new discoveries and surprises. During randomization, it considers the user's past viewing history to prevent the same story from being displayed too frequently.

The Customization Unit is implemented as a user interface on the terminal. This unit provides functionality in the paid version for users to generate summaries by specifying particular individuals or time periods. For example, if a user wishes to review conversations with a specific friend or interactions from a particular month, they can set conditions using dropdown menus or calendar widgets. When the user sets conditions, this information is sent to the server, and a summary is generated based on the specified conditions. This enables users to perform flexible reviews tailored to their interests and needs.

Thus, the system according to this embodiment provides users with an efficient and emotionally resonant reflection experience through the cooperative operation of its various components. The seamless integration of the entire system enables the provision of flexible services tailored to user needs.

### Implementation Steps

Step 1: Data Collection

Message data is collected via the API of the messenger app installed on the user's device, with the user's permission. When the user selects a specific chat thread, its history is retrieved, collecting information including metadata such as text messages, sender ID, recipient ID, and timestamps. For example, if the user wishes to review past conversations with a friend, the history of that conversation is collected. The collected data is temporarily stored within the device, encrypted using encryption techniques such as AES, and transmitted to the server via a secure communication protocol.

### Step 2: Data Analysis

The encrypted data received on the server is decrypted and analyzed using natural language processing technology. A morphological analysis engine is used to parse the grammatical structure of each message and identify parts of speech such as nouns, verbs, and adjectives. Frequently occurring words and phrases within the conversation are extracted to grasp the conversation's theme. Additionally, sentiment analysis technology is used to evaluate the emotional tone of each message, assigning sentiment labels such as positive, negative, or neutral. Furthermore, a keyword extraction algorithm is applied to identify words related to specific events or emotions, thereby pinpointing the central topic of the conversation.

### Step 3: Summary Generation

Operating on the server, it generates a summary based on information obtained by the data analysis unit. Using a generative AI model, it understands the flow of the conversation and extracts key points. The AI model emphasizes particularly emotional moments or points where important decisions were made within the conversation, generating narrative-style text. For example, if users are discussing a specific event, it constructs the narrative around messages related to that even, emphasizes particularly emotional moments or points where important decisions were made within the conversation, generating narrative-style text. For example, if users are discussing a specific event, it constructs a story centered around messages related to that event. Furthermore, the AI considers the flow of the conversation from start to finish to form a natural storyline. Examples of prompts fed to the generative AI include: "Highlight the most emotional moments in this conversation and summarize them as a story," or "Extract the key points from this conversation and express them in narrative form."

### Step 4: Randomization

This function randomly selects generated summaries and runs on the server. It uses a random number generation algorithm to randomly choose which summary to present to the user. For example, displaying a different story each time the user opens the app provides new discoveries and surprises. During randomization, the user's past viewing history is considered to prevent the same story from appearing too frequently.

### Step 5: Customization

Implemented as a user interface on the device. In the paid version, it provides functionality for users to generate summaries by specifying particular individuals or time periods. For instance, if a user wishes to review conversations with a specific friend or interactions from a particular month, they can set conditions using dropdown menus or calendar widgets. When the user sets these conditions, the information is sent to the server, and a summary is generated based on the specified criteria. This enables users to perform flexible reviews tailored to their interests and needs.

### Specific Use Cases

For example, consider a user engaging in daily conversations with friends using a messenger app. This user wishes to review conversations from a specific period, particularly seeking to revisit emotional moments or significant events. In this case, the user selects a specific chat thread within the messenger app, and the data collection component gathers that history. The collected data is sent to the server as metadata containing text messages, sender ID, recipient ID, and timestamps. The collected data is sent to the server as metadata, including text messages, sender ID, recipient ID, and timestamps.

On the server, the data analysis unit analyzes the message data using natural language processing technology. Through morphological analysis, it understands the grammatical structure of each message and extracts frequently occurring words and phrases. Using sentiment analysis technology, it evaluates the emotional tone of each message and assigns sentiment labels such as positive, negative, or neutral. This enables the identification of conversation themes and shifts in sentiment.

The summary generation unit uses a generative AI model to generate summaries based on the analyzed information. The AI understands the flow of the conversation and generates narrative-style text that emphasizes particularly emotional moments or points where important decisions were made. Specific examples of prompt sentences fed to the generative AI include: "Highlight the most emotional moments in this conversation and summarize it as a story," or "Extract the key points of this conversation and express them in a narrative format."

The randomization unit randomly selects generated summaries and provides them to the user. This allows users to enjoy different stories each time they open the app, offering new discoveries and surprises. By considering the user's past viewing history and preventing the same story from appearing too frequently, it consistently delivers a fresh experience.

Furthermore, the customization unit provides functionality for users to generate summaries by specifying particular individuals or time periods. Users set conditions using dropdown menus or calendar widgets, and this information is sent to the server. Summaries are generated based on the specified conditions, enabling users to perform flexible reflections tailored to their interests and needs.

Thus, the present invention enables users to efficiently and emotionally reflect on past conversations, aiming to deepen emotional connections. The entire system works seamlessly together, enabling flexible services tailored to user needs.

### Application Example 1

The flow of specific processing in Application Example 1 is described below. The components of the system described below are implemented by the data processing device 12 and the smart device 14. The data processing device 12 is referred to as the "server," and the smart device 14 is referred to as the "terminal."

### Implementation Examples for the Present Invention

A more specific and detailed description of an embodiment for implementing the present invention is provided below. This embodiment is a system that records communication and activities between users and staff within a nursing care facility, summarizes this information, and provides it. The specific functions and processing of each component are described in detail below.

First, the data collection unit is described. This unit collects conversations and activities between residents and staff in real time using voice recognition devices and activity sensors installed within the care facility. Voice recognition devices are placed in each room and common area of the facility to capture conversations between residents and staff with high accuracy. The audio data is converted into text data in real time and immediately transmitted to the server. For example, if a resident tells a staff member, "I feel a little sluggish today," that content is instantly converted to text and recorded in the database. Activity sensors are also installed throughout the facility to record residents' movements and activities. These sensors record detailed information such as the user's walking distance, exercise volume during rehabilitation, and meal intake status. For example, sensors detect and collect data on the distance a user walks within the facility, as well as the type and number of exercises performed during rehabilitation. This enables detailed understanding of the user's activity patterns and health status.

Next, the data analysis unit runs on the server and analyzes the collected data. This unit uses natural language processing technology to analyze text data and extract important information from conversation content. For example, if a user reports a change in physical condition, it identifies that information and utilizes it for health management. Furthermore, it employs sentiment analysis technology to evaluate the emotional tone of each message, assigning sentiment labels such as positive, negative, or neutral. This enables understanding the user's emotional state and adjusting care plans as necessary. Furthermore, for activity data, it analyzes the user's movement patterns and activity levels to aid in assessing health status. For example, if activity levels are lower than usual, it generates an alert suggesting possible illness. This allows caregivers to take prompt countermeasures.

The summary generation unit synthesizes the user's daily activities and health status based on the analyzed information. This unit uses generative AI to extract key points and present them in a format easily understandable to caregivers and family members. For example, it generates summaries such as: "The user took a 30-minute walk in the morning and performed one hour of rehabilitation in the afternoon. Their condition is good, with no particular issues observed. “Examples of prompts fed to the generative AI include: "Summarize the user's daily activities from this data and provide a concise report on their health status," or "Highlight the most emotionally charged moment in this conversation and summarize it as a narrative." This enables caregivers and family members to efficiently grasp the user's condition and revise care plans as necessary.

Finally, the notification unit notifies caregivers and family members of the generated summary. This unit enables family members to check the user's status remotely via smartphones or tablets. For example, family members can view summaries through a dedicated app and send feedback to caregivers as needed. Furthermore, if an abnormality is detected, it immediately issues an alert, prompting swift action. This allows family members to monitor the user's status with peace of mind, while enabling caregivers to respond quickly and appropriately.

Thus, the present invention aims to efficiently monitor the condition of users within care facilities and improve the quality of care. The entire system operates seamlessly, enabling flexible services tailored to user needs.

### System Configuration

The system according to this embodiment comprises a data collection unit, a data analysis unit, a summary generation unit, and a notification unit. The data collection unit has the function of collecting conversations and activities between residents and staff in real time using voice recognition devices and activity sensors installed within the care facility. Voice recognition devices are installed in each room and common area within the facility, capturing conversations between residents and staff with high accuracy. For example, if a resident tells a staff member, "I feel a little sluggish today," that content is immediately transcribed into text and recorded in the database. Activity sensors are also installed throughout the facility to record residents' movements and activities. These sensors meticulously record details such as walking distance, exercise volume during rehabilitation, and meal intake status. For instance, sensors detect and collect data on the distance a resident walk within the facility, as well as the type and number of exercises performed during rehabilitation. This enables detailed understanding of residents' activity patterns and health status.

The data analysis unit runs on the server and possesses the capability to analyze collected data. This unit analyzes text data using natural language processing technology to extract important information from conversation content. For example, if a user reports a change in physical condition, it identifies that information to aid in health management. Furthermore, it uses sentiment analysis technology to evaluate the emotional tone of each message and assigns sentiment labels such as positive, negative, or neutral. This enables understanding the user's emotional state and adjusting care plans as necessary. Furthermore, for activity data, it analyzes the user's movement patterns and activity levels to aid in assessing health status. For example, if activity levels are lower than usual, it generates an alert suggesting possible illness. This allows caregivers to take prompt countermeasures.

The summary generation unit has the function of summarizing the user's daily activities and health status based on the analyzed information. This unit uses generative AI to extract key points and present them in a format easily understandable to caregivers and family members. For example, it generates summaries such as: "The user took a 30-minute walk in the morning and performed one hour of rehabilitation in the afternoon. Their condition is good, with no particular issues observed. “Examples of prompts fed to the generative AI include: "Summarize the user's daily activities from this data and provide a concise report on their health status," or "Highlight the most emotional moment in this conversation and summarize it as a narrative." This enables caregivers and family members to efficiently grasp the user's condition and revise care plans as needed.

The notification unit has the function of notifying caregivers and family members of the generated summaries. This unit enables family members to check the user's status remotely via smartphones or tablets. For example, family members can view the summaries through a dedicated app and send feedback to caregivers as needed. Furthermore, if an abnormality is detected, it immediately issues an alert to prompt a swift response. This allows family members to monitor the user's condition with peace of mind, while caregivers can respond quickly and appropriately.

Thus, the system according to this embodiment aims to efficiently grasp the condition of users within the care facility through the cooperation of each component, thereby improving the quality of care. The entire system enables seamless coordination, allowing for flexible services tailored to user needs.

### Implementation Steps

### Step 1: Data Collection

Conversations and activities between residents and staff are collected in real time using voice recognition devices and activity sensors installed within the care facility. Voice recognition devices are placed in each room and common area of the facility to capture conversations between residents and staff with high accuracy. For example, if a resident tells a staff member, "I feel a bit sluggish today," that statement is immediately transcribed into text and recorded in the database. Activity sensors are also installed throughout the facility to record residents' movements and activities. These sensors meticulously log details such as walking distance, exercise volume during rehabilitation, and meal intake status. For instance, sensors detect and collect data on the distance a resident walked within the facility, as well as the type and number of exercises performed during rehabilitation.

### Step 2: Data Analysis

The data analysis component, executed on the server, analyzes the collected data. This component uses natural language processing technology to analyze text data and extract important information from conversation content. For example, if a user reports a change in physical condition, this information is identified and utilized for health management. Furthermore, sentiment analysis technology is used to evaluate the emotional tone of each message, assigning emotional labels such as positive, negative, or neutral. This enables understanding of the user's emotional state and allows for adjustments to care plans as needed. Additionally, for activity data, the user's movement patterns and activity levels are analyzed, such as positive, negative, or neutral. This allows the system to understand the user's emotional state and adjust care plans as needed. Furthermore, for activity data, the system analyzes the user's movement patterns and activity levels to help assess their health status. For example, if activity levels are lower than usual, it generates an alert suggesting possible health issues.

### Step 3: Summary Generation

Based on the analyzed information, summarize the user's daily activities and health status. In this step, generative AI is used to extract key points and present them in a format easily understandable for caregivers and family members. For example, it generates summaries such as: "The user took a 30-minute walk in the morning and performed one hour of rehabilitation in the afternoon. Their condition is good, with no particular issues observed. “Examples of prompts fed to the generative AI include: "Summarize the user's daily activities from this data and provide a concise report on their health status," or "Highlight the most emotional moment in this conversation and summarize it as a narrative."

### Step 4: Notification

Notify caregivers and family members of the generated summary. This step enables family members to check the user's status remotely via smartphones or tablets. For example, family members can view the summary via a dedicated app and send feedback to caregivers as needed. Furthermore, if an anomaly is detected, an immediate alert is issued to prompt swift action. This allows family members to monitor the user's condition with peace of mind, while enabling caregivers to respond quickly and appropriately.

### Specific Use Cases

For example, consider a system for care facilities that details users' daily activities and interactions with staff, providing this information to family members and caregivers. This system aims to efficiently monitor users' health status and activity levels, thereby improving the quality of care.

Voice recognition devices are installed in each room and common areas within the care facility, capturing conversations between residents and staff in real time. For example, if a resident tells a staff member, "I feel a bit sluggish today," that statement is immediately transcribed into text and recorded in a database. This information aids in managing the resident's physical condition. Additionally, activity sensors meticulously record residents' movements and activities. For instance, sensors detect and collect data on the distance a resident walks within the facility, as well as the type and number of exercises performed during rehabilitation. This enables detailed understanding of residents' activity patterns and health status.

The collected data is analyzed by the data analysis unit running on the server. Natural language processing technology analyzes text data to extract important information from conversation content. For example, if a user reports a change in physical condition, this information is identified and utilized for health management. Furthermore, sentiment analysis technology evaluates the emotional tone of each message, assigning sentiment labels such as positive, negative, or neutral. This allows for understanding the user's emotional state and adjusting care plans as necessary.

Based on the analyzed information, the summary generation unit summarizes the user's daily activities and health status. Using generative AI, it extracts key points and presents them in a format easily understandable for caregivers and family members. For example, it generates summaries such as: "The user took a 30-minute walk in the morning and underwent one hour of rehabilitation in the afternoon. Their condition is good, with no particular issues observed. “Examples of prompts fed to the generative AI include: "Summarize the user's daily activities from this data and provide a concise report on their health status," or "Highlight the most emotionally charged moment in this conversation and summarize it as a narrative."

The generated summary is notified to caregivers and family members via the notification section. Family members can check the user's status remotely via smartphones or tablets. For example, family members can view the summary via a dedicated app and send feedback to caregivers as needed. Furthermore, if an abnormality is detected, an immediate alert is issued to prompt a swift response. This allows family members to monitor the user's condition with peace of mind, while enabling caregivers to respond quickly and appropriately. Thus, the present invention aims to efficiently monitor the condition of users within care facilities and improve the quality of care.

The specific processing unit 290 transmits the results of the specific processing to the smart device 14. On the smart device 14, the control unit 46A instructs the output device 40 to output the results of the specific processing. The microphone 38B acquires audio indicating user input regarding the results of the specific processing. The control unit 46A transmits the audio data indicating the user input acquired by the microphone 38B to the data processing unit 12. At the data processing unit 12, the specific processing unit 290 acquires the audio data.

The data generation model 58 is a so-called generative AI (Artificial Intelligence). An example of the data generation model 58 is ChatGPT (registered trademark) (Internet search <URL: https://openai.com/blog/chatgpt>). The data generation model 58 is obtained by performing deep learning on a neural network. The data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58. The data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. The AI may also be an AI agent. Furthermore, when the processing of the aforementioned components is performed by the AI, such processing may be performed in part or in whole by the AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, which is then processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the smart device 14.

### Second Embodiment

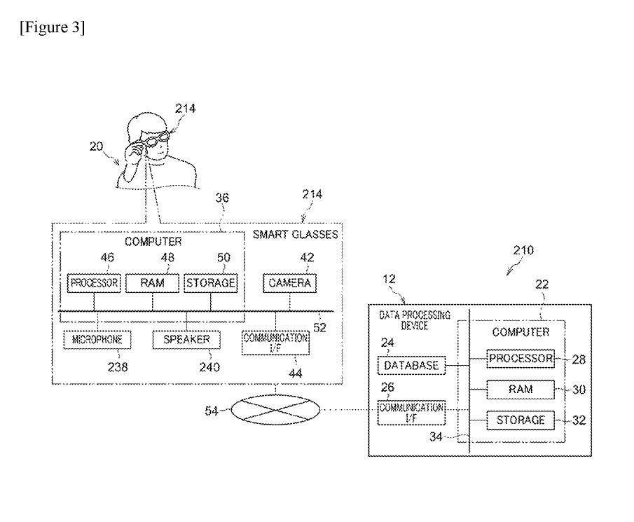

FIG. 3 shows an example configuration of the data processing system 210 according to the second embodiment.

As shown in FIG. 3, the data processing system 210 includes a data processing device 12 and smart glasses 214. An example of the data processing device 12 is a server.

The data processing device 12 includes a computer 22, a database 24, and a communication I/F 26. The computer 22 is an example of a "computer" according to the technology of the present disclosure. The computer 22 includes a processor 28, RAM 30, and storage 32.The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The smart glasses 214 include a computer 36, a microphone 238, a speaker 240, a camera 42, and a communication I/F 44. The computer 36 includes a processor 46, RAM 48, and storage 50. Processor 46, RAM 48, and storage 50 are connected to bus 52. Microphone 238, speaker 240, and camera 42 are also connected to bus 52.

Microphone 238 receives voice input from user 20 to accept instructions or other commands. Microphone 238 captures the voice input from user 20, converts the captured voice into audio data, and outputs it to processor 46. Speaker 240 outputs audio in accordance with instructions from processor 46.

The camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surroundings (e.g., an imaging range defined by a field of view equivalent to that of a typical healthy person).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

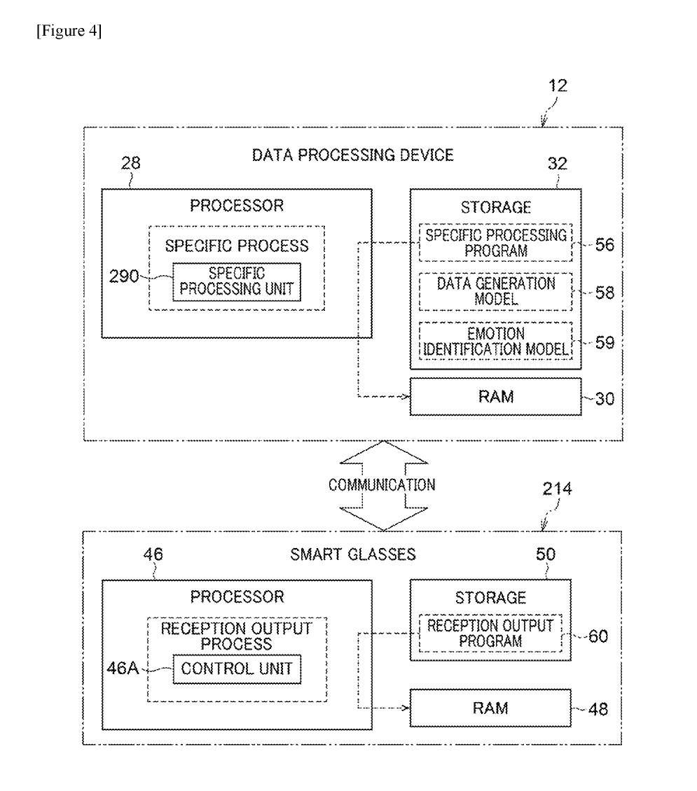

FIG. 4 shows an example of the main functions of the data processing device 12 and the smart glasses 214. As shown in FIG. 4, specific processing is performed by the processor 28 in the data processing device 12. The specific processing program 56 is stored in the storage 32.

The specific processing program 56 is an example of a "program" related to the technology of this disclosure. The processor 28 reads the specific processing program 56 from the storage 32 and executes the read specific processing program 56 on the RAM 30. The specific processing is realized by the processor 28 operating as the specific processing unit 290 according to the specific processing program 56 executed on the RAM 30.

Storage 32 stores a data generation model 58 and an emotion identification model 59. The data generation model 58 and emotion identification model 59 are used by specific processing unit 290. Specific processing unit 290 can estimate a user's emotion using emotion identification model 59 and perform specific processing using the user's emotion. The emotion estimation function (emotion identification function) using the emotion identification model 59 performs various estimations and predictions concerning the user's emotion, including estimation and prediction of the user's emotion, but is not limited to such examples. Furthermore, estimation and prediction of emotion also includes, for example, analysis (parsing) of emotion.

In the smart glasses 214, the processor 46 performs the reception output processing. The reception output program 60 is stored in the storage 50. The processor 46 reads the reception output program 60 from the storage 50 and executes the read reception output program 60 on the RAM 48. The reception output processing is realized by the processor 46 operating as the control unit 46A according to the reception output program 60 executed on the RAM 48. The reception output processing is performed by the processor 46 acting as a control unit 46A according to the reception output program 60 executed on RAM 48. Note that the smart glasses 214 may also have a data generation model 58 and an emotion identification model 59, and can perform processing similar to that of the identification processing unit 290 using these models.

Next, the identification processing performed by the identification processing unit 290 of the data processing device 12 is described. The components of the system described below are implemented by the data processing device 12 and the smart glasses 214. In the following description, the data processing device 12 is referred to as the "server," and the smart glasses 214 are referred to as the "terminal."

### Example 1

The flow of the specific processing is the same as that described in Example 1 of the first embodiment, so the description is omitted.

### Application Example 1

The flow of the specific processing in Example 1 described in the first embodiment is the same as above, so the explanation is omitted.

The specific processing unit 290 transmits the result of the specific processing to the smart glasses 214. In the smart glasses 214, the control unit 46A causes the speaker 240 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

The data generation model 58 is a so-called generative AI (Artificial Intelligence). An example of the data generation model 58 is ChatGPT (registered trademark) (Internet search <URL: https://openai.com/blog/chatgpt>). The data generation model 58 is obtained by performing deep learning on a neural network. The data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while using the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58. The data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. The AI may also be an AI agent. Furthermore, when the processing of the aforementioned components is performed by the AI, such processing may be performed in part or in whole by the AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit may acquire step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using the generation AI. For example, the provision unit is implemented by the output device 40 of the smart device 14 or the specific processing unit 290 of the data processing device 12 and provides the generated cooking menu to the user. The correspondence between each unit and the device or control unit is not limited to the examples described above and can be modified in various ways.

The above embodiment described a form where specific processing is performed by the data processing device 12, but the technology disclosed herein is not limited thereto; specific processing may also be performed by the smart glasses 214.

### Third Embodiment

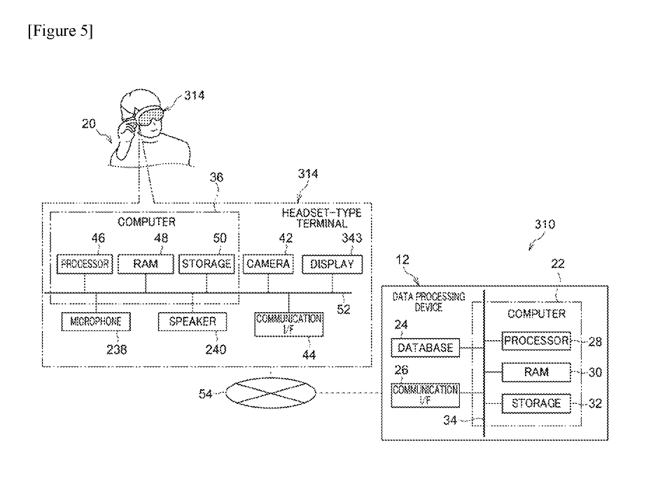

FIG. 5 shows an example configuration of the data processing system 310 according to the third embodiment.

As shown in FIG. 5, the data processing system 310 includes a data processing device 12 and a headset-type terminal 314. An example of the data processing device 12 is a server.

The data processing device 12 includes a computer 22, a database 24, and a communication I/F 26. The computer 22 is an example of a "computer" according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The headset-type terminal 314 comprises a computer 36, a microphone 238, a speaker 240, a camera 42, a communication interface 44, and a display 343. The computer 36 includes a processor 46, RAM 48, and storage 50. The processor 46, RAM 48, and storage 50 are connected to a bus 52. The microphone 238, speaker 240, camera 42, and display 343 are also connected to the bus 52.

Microphone 238 receives voice input from user 20 to accept instructions or other commands. Microphone 238 captures the voice input from user 20, converts the captured voice into audio data, and outputs it to processor 46. Speaker 240 outputs audio in accordance with instructions from processor 46.

The camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surroundings (e.g., within a field of view equivalent to that of a typical healthy individual).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

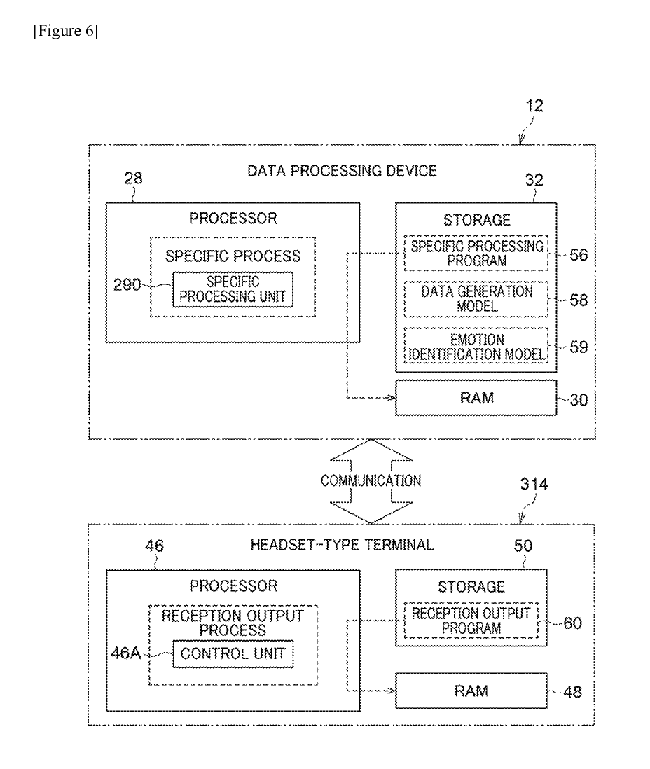

FIG. 6 shows an example of the main functions of the data processing device 12 and the headset-type terminal 314. As shown in FIG. 6, specific processing is performed by the processor 28 in the data processing device 12. The specific processing program 56 is stored in the storage 32.

The specific processing program 56 is an example of a "program" related to the technology of this disclosure. The processor 28 reads the specific processing program 56 from the storage 32 and executes the read specific processing program 56 on the RAM 30. The specific processing is realized by the processor 28 operating as the specific processing unit 290 according to the specific processing program 56 executed on the RAM 30.

Storage 32 stores a data generation model 58 and an emotion identification model 59. The data generation model 58 and the emotion identification model 59 are used by specific processing unit 290.

In the headset-type terminal 314, reception output processing is performed by the processor 46. The reception output program 60 is stored in the storage 50. Processor 46 reads the reception output program 60 from storage 50 and executes the read reception output program 60 on RAM 48. Reception output processing is achieved by processor 46 operating as control unit 46A according to the reception output program 60 executed on RAM 48.

Next, the specific processing performed by the specific processing unit 290 of the data processing device 12 is described. The various parts of the system described below are implemented by the data processing device 12 and the headset-type terminal 314. In the following description, the data processing device 12 is referred to as the "server," and the headset-type terminal 314 is referred to as the "terminal."

### Example 1

The flow of the specific processing is the same as that described in Example 1 of the first embodiment, so the description is omitted.

### Application Example 1

The flow of the specific processing in Example 1 described in the first embodiment is the same as described above, so the explanation is omitted.

The specific processing unit 290 transmits the result of the specific processing to the headset-type terminal 314. At the headset-type terminal 314, the control unit 46A causes the speaker 240 and the display 343 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

The data generation model 58 is a so-called generative AI (Artificial Intelilgence). An example of the data generation model 58 is ChatGPT (registered trademark) (Internet search <URL: https://openai.com/blog/chatpgt>). The data generation model 58 is obtained by performing deep learning on a neural network. The data generation model 58 receives input prompts containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats such as audio data, text data, and image data. The data generation model 58 may include, for example, text generation AI, image generation AI, multimodal generation AI, etc. Here, inference refers to, for example, analysis, classification, prediction, and/or summarization. The specific processing unit 290 performs the specific processing described above using the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others and can perform various processing tasks, but are not limited to these examples. Furthermore, the AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the acquisition unit is implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12. However, the technology disclosed herein is not limited to this, and specific processing may also be performed by the headset-type terminal 314.

### Fourth Embodiment

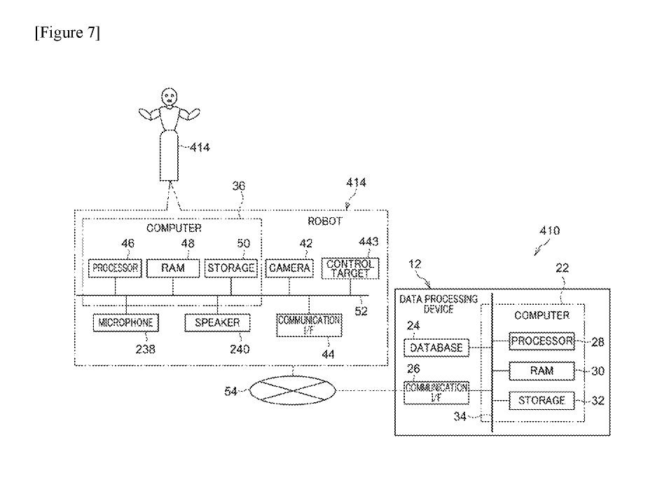

FIG. 7 shows an example configuration of the data processing system 410 according to the fourth embodiment.

As shown in FIG. 7, the data processing system 410 includes a data processing device 12 and a robot 414. An example of the data processing device 12 is a server.

The data processing device 12 includes a computer 22, a database 24, and a communication I/F 26. The computer 22 is an example of a "computer" related to the technology of this disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

Robot 414 includes a computer 36, a microphone 238, a speaker 240, a camera 42, a communication I/F 44, and a control target 443. Computer 36 includes a processor 46, RAM 48, and storage 50. Processor 46, RAM 48, and storage 50 are connected to bus 52. Furthermore, microphone 238, speaker 240, camera 42, and controlled object 443 are also connected to bus 52.

Microphone 238 receives voice input from user 20 to accept instructions or other commands. Microphone 238 captures the voice input from user 20, converts the captured voice into audio data, and outputs it to processor 46. Speaker 240 outputs audio in accordance with instructions from processor 46.

The camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surroundings (e.g., an imaging range defined by a field of view equivalent to that of a typical healthy person).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

The control target 443 includes a display device, LEDs for the eye section, and motors for driving the arms, hands, legs, etc. The posture and gestures of robot 414 are controlled by controlling the motors for the arms, hands, legs, etc. Part of the robot 414's emotions can be expressed by controlling these motors. Furthermore, the robot 414's facial expressions can also be expressed by controlling the light emission state of the LEDs in its eyes.

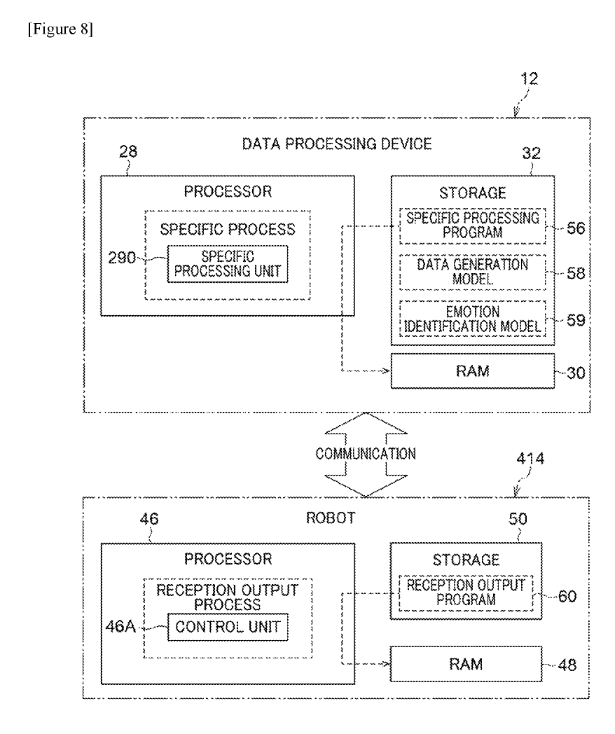

FIG. 8 shows an example of the main functions of the data processing device 12 and the robot 414. As shown in FIG. 8, specific processing is performed by the processor 28 in the data processing device 12. The specific processing program 56 is stored in the storage 32.

The specific processing program 56 is an example of a "program" related to the technology of this disclosure. The processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and the emotion identification model 59 are used by the specific processing unit 290.

In robot 414, reception output processing is performed by processor 46. Storage 50 stores a reception output program 60. Processor 46 executes the reception output program 60 stored in storage 50 and execute the read reception output program 60 in RAM 48. The reception output processing is realized by the processor 46 operating as the control unit 46A according to the reception output program 60 executed in RAM 48.

Next, the specific processing performed by the specific processing unit 290 of the data processing device 12 is described. The various parts of the system described below are implemented by the data processing device 12 and the robot 414. In the following description, the data processing device 12 is referred to as the "server," and the robot 414 is referred to as the "terminal."

### Example 1

The flow of the specific processing is the same as that described in Example 1 of the first embodiment, so the description is omitted.

### Application Example 1

The flow of the specific processing in Example 1 described in the first embodiment is the same as described above, so the explanation is omitted.

The specific processing unit 290 transmits the result of the specific processing to the robot 414. In the robot 414, the control unit 46A causes the speaker 240 and the control target 443 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

The data generation model 58 is a so-called generative AI (Artificial Intelligence). An example of the data generation model 58 is ChatGPT (registered trademark) (Internet search <URL: https://openai.com/blog/chatgpt>). The data generation model 58 is obtained by performing deep learning on a neural network. The data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like include multiple types of data generation models 58, and the data generation model 58 includes AI other than generative AI. AI other than generative AI includes, for example, linear regression, logistic regression, decision trees, random forests, support vector machines (SVM), k-means clustering, convolutional neural networks (CNNs), recurrent neural networks (RNN), generative adversarial networks (GAN), or naive Bayes, among others. AI may also be an AI agent. Furthermore, when processing by the aforementioned components is performed by AI, such processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the acquisition unit is implemented by the control unit 46A of the smart device 14 or the specific processing unit 290 of the data processing device 12.For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the robot 414.

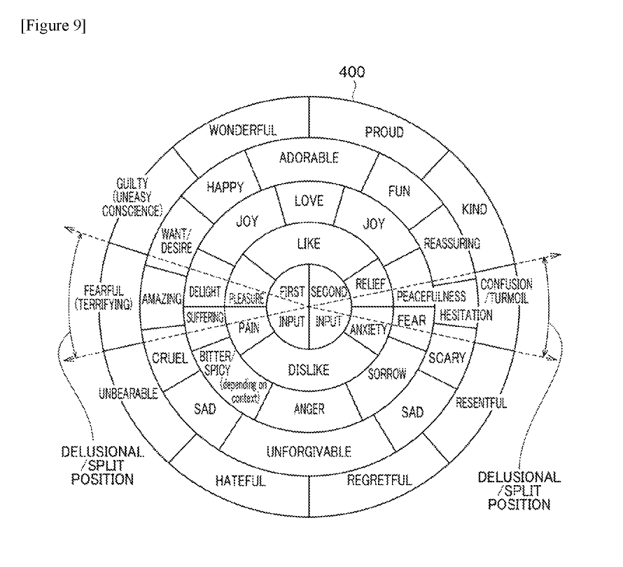

The emotion identification model 59, functioning as an emotion engine, may determine the user's emotion according to a specific mapping. Specifically, the emotion identification model 59 may determine the user's emotion according to an emotion map (see FIG. 9), which is a specific mapping. Furthermore, the emotion identification model 59 may similarly determine the robot's emotion, and the specific processing unit 290 may perform specific processing using the robot's emotion.

FIG. 9 is a diagram showing an emotion map 400 where multiple emotions are mapped. In the emotion map 400, emotions are arranged radially in concentric circles from the center. Emotions closer to the center of the concentric circles represent more primitive states. Emotions representing states or behaviors arising from mental states are arranged further outward on the concentric circles. Emotion is a concept encompassing affect and mental states. Generally, emotions generated from reactions occurring within the brain are placed on the left side of the concentric circles. Generally, emotions induced by situational judgment are placed on the right side of the concentric circles. Generally, emotions generated from reactions occurring within the brain and also induced by situational judgment are placed on the upper and lower sides of the concentric circles. Furthermore, the upper part of the concentric circle contains "pleasant" emotions, while the lower part contains "unpleasant" emotions. Thus, the Emotion Map 400 maps multiple emotions based on the structure of their origin, with emotions that tend to occur simultaneously mapped close together.

These emotions are distributed around the 3 o'clock position on Emotion Map 400, typically oscillating between feelings of security and anxiety. In the right half of Emotion Map 400, situational awareness takes precedence over internal sensations, resulting in a calmer impression.

The inner part of the emotion map 400 represents the mind, while the outer part represents behavior. Therefore, the further out on the emotion map 400, the more visible the emotion becomes (manifesting in behavior).

Here, human emotions are based on various balances, such as posture and blood sugar levels. When these balances deviate from the ideal, it indicates a state of discomfort; when they approach the ideal, it indicates a state of comfort. Similarly, for robots, automobiles, motorcycles, etc., emotions can be created based on various balances, such as posture and battery level. When these balances deviate from the ideal, it indicates a state of discomfort; when they approach the ideal, it indicates a state of comfort. Emotion maps, for example, Dr. Mitsuyoshi's Emotion Map (Research on Speech Emotion Recognition and Neurophysiological Signal Analysis of Emotions, Tokushima University, Doctoral Dissertation: https://ci.nii.ac.jp/naid/500000375379). The left half of the emotion map displays emotions belonging to the "Reaction" area, where sensory input dominates. The right half of the emotion map displays emotions belonging to the "Situation" domain, where situational awareness is dominant.

The emotion map defines two emotions that promote learning. One is the negative emotion around the center of the "repentance" or "reflection" area on the situation side. That is, when the robot experiences negative emotions like "I never want to feel this way again" or "I don't want to be scolded anymore. "The other is the positive emotion around "desire" on the reaction side. That is, when the robot feels positive emotions like "I want more" or "I want to know more."

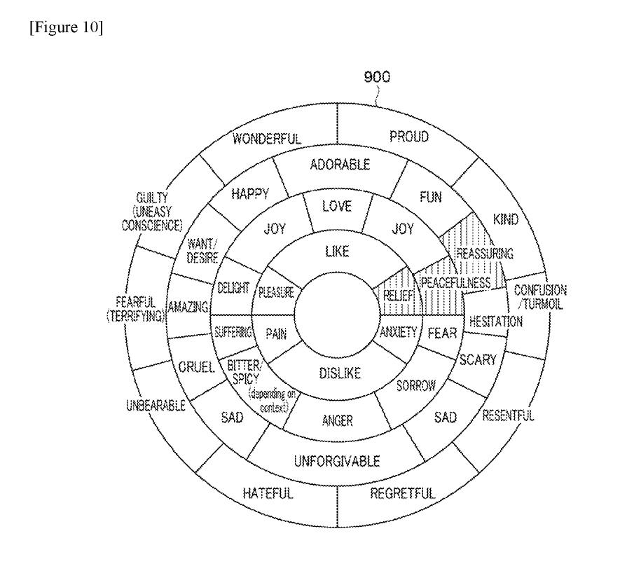

The emotion identification model 59 inputs the user input into a pre-trained neural network, obtains emotion values corresponding to each emotion shown in the emotion map 400, and determines the user's emotion. This neural network is pre-trained based on multiple training data sets, which are combinations of user input and emotion values corresponding to each emotion shown in the emotion map 400. Furthermore, this neural network is trained such that emotions positioned close to each other, as shown in the emotion map 900 in FIG. 10, have similar values. FIG. 10 illustrates an example where multiple emotions, such as "reassurance," "tranquility," and "confidence," have similar emotion values.

The above description primarily explains the system according to the present disclosure in terms of the functions of the data processing device 12. However, the system according to the present disclosure is not necessarily implemented on a server. The system according to the present disclosure may be implemented as a general information processing system. For example, the present disclosure may be implemented as a software program operating on a personal computer or as an application operating on a smartphone or the like. The method according to the present disclosure may be provided to users in a SaaS (Software as a Service) format.

The above embodiment illustrated an example configuration where specific processing is performed by a single computer 22. However, the technology of this disclosure is not limited thereto. Distributed processing may be performed by multiple computers, including computer 22, for specific processing. For example, data generation model 58 may be provided in an external device of data processing device 12, and data generation corresponding to input data may be performed in said external device. For example, the data generation model 58 may be provided in an external device of the data processing device 12, and data generation corresponding to input data may be performed in said external device.

The above embodiment described a configuration where the specific processing program 56 is stored in the storage 32. However, the technology disclosed herein is not limited to this. For example, the specific processing program 56 may be stored on a portable, computer-readable non-volatile storage medium, such as a USB (Universal Serial Bus) memory. The specific processing program 56 stored on the non-volatile storage medium is installed on the computer 22 of the data processing device 12. The processor 28 executes specific processing according to the specific processing program 56.

Alternatively, the specific processing program 56 may be stored on a storage device, such as a server, connected to the data processing device 12 via the network 54. Upon request from the data processing device 12, the specific processing program 56 is downloaded and installed on the computer 22.

It should be noted that it is not necessary to store the entire specific processing program 56 on a storage device such as a server connected to the data processing device 12 via the network 54, or to store the entire specific processing program 56 in the storage 32. It is also possible to store only a portion of the specific processing program 56.

Various types of processors can be used as hardware resources to execute the specific processing. Examples of processors include a CPU, which is a general-purpose processor that functions as a hardware resource for executing specific processing by executing software, i.e., a program. Additionally, processors may include dedicated electronic circuits, such as FPGAs (Field-Programmable Gate Array), PLDs (Programmable Logic Device), or ASICs (Application Specific Integrated Circuit), which are processors with circuit configurations specifically designed to execute particular processing tasks. Each processor incorporates or connects to memory, and each processor executes specific processing by utilizing this memory.

The hardware resources for executing specific processing may be comprised of one of these various processors, or may be comprised of a combination of two or more processors of the same or different types (e.g., a combination of multiple FPGAs, or a combination of a CPU and an FPGA). Furthermore, the hardware resources for executing specific processing may be comprised of a single processor.

Examples of configurations using a single processor include, first, a form where one processor is composed of a combination of one or more CPUs and software, functioning as a hardware resource that executes specific processing. Second, there is a form that uses a processor, such as a System-on-a-chip (SoC), which implements the entire system's functionality—including multiple hardware resources for executing specific processing—on a single IC chip. Thus, specific processing is realized as hardware resources using one or more of the aforementioned types of processors.

Furthermore, regarding the hardware structure of these various processors, more specifically, electrical circuits combining circuit elements such as semiconductor devices can be used. Also, the specific processing described above is merely one example. Therefore, it goes without saying that within the scope not deviating from the main purpose, unnecessary steps may be omitted, new steps may be added, or the processing order may be changed.

The above description and illustrations provide a detailed explanation of the aspects pertaining to the technology of this disclosure and represent merely one example of the technology disclosed herein. For example, the explanations regarding the configuration, functions, operations, and effects described above are examples of the configuration, functions, operations, and effects pertaining to the aspects of the technology disclosed herein. Therefore, it goes without saying that within the scope of not deviating from the essence of the technology of this disclosure, unnecessary portions may be deleted, new elements may be added, or replacements may be made to the above-described content and illustrated content. Furthermore, to avoid complexity and facilitate understanding of the part pertaining to the technology of this disclosure, descriptions of technical common knowledge, etc., that are particularly unnecessary for enabling the implementation of the technology of this disclosure have been omitted from the above-described content and illustrated content.

All literature, patent applications, and technical specifications cited herein are incorporated by reference to the same extent as if each individual literature, patent application, and technical specification were specifically and individually cited herein.

With regard to the embodiments described above, the following Supplementary notes are further disclosed.

### Supplementary note 1

A system comprising a data collection unit, a data analysis unit, a summary generation unit, and a notification unit. The data collection unit has the function of collecting conversations and activities between users and staff in real time using voice recognition devices and activity sensors installed within a care facility, and recording them as text data and activity data. The data analysis unit has the function of analyzing the collected data using natural language processing technology and sentiment analysis technology to understand the content of conversations and the health status of users. The summary generation unit utilizes generative AI based on the analyzed information to summarize the user's daily activities and health status, providing it in a format easily understandable to caregivers and family members. The notification unit notifies caregivers and family members of the generated summary, enabling them to check the user's status remotely via smartphones or tablets.

### Supplementary note 2

The system described in Supplementary note 1, wherein the data collection unit uses voice recognition devices installed within the care facility to transcribe conversations between users and staff in real time, and uses activity sensors to record the user's movements and activities. This enables immediate recording of any changes in the user's condition reported to staff as text data, and allows the activity sensors to accurately record the user's exercise volume and distance traveled.

### Supplementary note 3

The system described in Supplementary note 1, wherein the summary generation unit utilizes generative AI to summarize the resident's daily activities and health status from the analyzed information, generating narrative-style text that emphasizes particularly emotional moments or significant events. Prompts fed to the generative AI include: "Summarize the user's daily activities from this data and provide a concise report on their health status" or "Highlight the most emotional moments within this conversation and summarize them as a narrative."

### Explanation of Symbols

10, 210, 310, 410 Data Processing System

12 Data Processing Device

14 Smart Device

214 Smart Glasses

314 Headset-type devices

414 Robot

What is claimed is:

1. A system comprising one or more processors and one or more non-transitory storage media storing instructions which, when executed by the one or more processors, cause the system to:
acquire, via an application programming interface of a messenger application and in response to user authorization, message data including text messages, sender identifiers, recipient identifiers, and timestamps;
encrypt the acquired message data and transmit the encrypted message data from a user terminal to a server via a secure communication protocol;
decrypt the encrypted message data at the server and perform natural language processing on the message data, the natural language processing including morphological analysis and keyword extraction to generate structured conversation data representing conversation themes and emotional attributes;
execute a generative artificial intelligence model on the structured conversation data to generate a narrative-style summary that reflects a temporal flow and emotional transitions of the conversation;
store a plurality of generated narrative-style summaries in a memory together with viewing history information associated with a user;
select, using a randomization algorithm that references the viewing history information, one of the plurality of generated narrative-style summaries so as to suppress repetitive presentation of an identical summary; and
output the selected narrative-style summary to a user interface of the user terminal,
wherein the above processing enables automated review of message histories in a manner not practically performable by manual human review.

2. The system of claim 1, wherein the encryption includes symmetric key encryption and the secure communication protocol includes Transport Layer Security (TLS).

3. The system of claim 1, wherein the natural language processing further includes sentiment analysis for assigning emotional labels to individual messages, and the generative artificial intelligence model generates the narrative-style summary by emphasizing message segments associated with changes in the emotional labels.

4. The system of claim 1, wherein the natural language processing generates an intermediate structured representation comprising message-level structural data and theme identifiers, and wherein the generative artificial intelligence model generates the narrative-style summary based on the intermediate structured representation rather than directly on the message data.

5. The system of claim 1, wherein the generative artificial intelligence model is executed using a prompt or control input generated based on results of the morphological analysis and keyword extraction.

6. The system of claim 1, wherein the viewing history information includes at least one of a frequency count, a last-viewed timestamp, or a recency score for each of the plurality of generated narrative-style summaries, and wherein the randomization algorithm weights selection probabilities based on the viewing history information.

7. The system of claim 1, further comprising receiving a user-specified condition identifying at least one of a conversation participant or a time period, and generating the narrative-style summary using only message data satisfying the user-specified condition.

8. The system of claim 1, wherein acquisition and encryption of the message data are performed on the user terminal, and the natural language processing and execution of the generative artificial intelligence model are performed on the server.

9. A robotic support apparatus comprising:
an emotion identification model comprising a neural network trained to estimate a user’s emotional state by mapping input values onto a concentric emotion map, where radial distance from the center represents a transition from primitive mental states to behavioral manifestations;
a generative AI unit configured to generate a response based on the estimated emotional state and a pre-registered health schedule; and
a robot control unit configured to execute a physical output by controlling at least one motor for driving a robotic limb or an LED eye state to express a robotic emotion corresponding to the user emotion.

10. The apparatus of claim 9, wherein the emotion map maps pleasant emotions to an upper portion and "unpleasant" emotions to a lower portion of the concentric circles.

11. The apparatus of claim 9, wherein the neural network is trained to assign similar emotion values to emotions positioned close to each other on the map, including reassurance and tranquility.

12. The apparatus of claim 9, wherein the processor is further configured to estimate a robotic internal emotional state based on a detected battery level or robotic posture.

13. The apparatus of claim 9, wherein the robot control unit selects a physical gesture based on whether the estimated emotion is categorized as a "reaction" or a "situation" on the emotion map.

14. The apparatus of claim 9, wherein the generative AI unit identifies a reported high body temperature from user speech and automatically triggers an electronic notification to medical staff.
